(19) United States
(12) Patent Application Publication
(10) Pub. No.: US 2024/0393276 A1
(43) Pub. Date: Nov. 28, 2024

(54) SENSOR DEVICE

(71) Applicant: SKF Lubrication Systems Germany GmbH, Walldorf (DE)

(72) Inventors: Juergen KREUTZKAEMPER, Waibstadt-Daisbach (DE); Stefan SCHUERMANN, Mühlhausen (DE); Holger MENGES, Walldorf (DE); Markus GREBE, Einhausen (DE); Henrik BUSE, Wörth am Rhein (DE); Dawid GLOWANIA, Mannheim (DE)

(21) Appl. No.: 18/667,089

(22) Filed: May 17, 2024

(30) Foreign Application Priority Data
May 25, 2023 (DE) 102023204896.5

(51) Int. Cl. G01N 27/02; G01N 33/28

(52) U.S. Cl. CPC G01N 27/028; G01N 33/2888

(57) ABSTRACT

A sensor device (4) detects a state of a lubricant. The sensor device (4) has a sensor unit (6). The sensor unit (6) is designed to measure the impedance of the lubricant according to the frequency of an applied alternating current. The sensor device (4) has an evaluation unit (8) which is designed to determine a state of the lubricant based on the measured impedance.

### CROSS-REFERENCE TO RELATED APPLICATION

This application claims priority to German Application No. 102023204896.5, filed May 25, 2023, the entirety of which is hereby incorporated by reference.

### FIELD

The present disclosure relates to a sensor device for detecting a state of a lubricant. Furthermore, the present disclosure relates to a lubrication system and a method for detecting a state of a lubricant.

### BACKGROUND

In lubrication systems which are used to deliver lubricant, e.g. oil or grease, to various parts or consumers to be lubricated, it is important to use a lubricant which is suitable for the respective application. Also, the lubricant must not be degraded, out of date or contaminated. Otherwise, this may lead to inadequate lubrication and, in the worst case, premature damage to the parts to be lubricated (e.g. bearings).

To prevent such inadequate lubrication or damage to the parts to be lubricated, it is necessary to regularly check and test the lubricant used. Through analysis of lubricants flowing back from a lubrication point, such checks also enable conclusions to be drawn about the state of the lubricant. For example, an increase in metal debris or reaction products may be identified.

Corresponding lubricant tests were hitherto only possible offline in the laboratory. To this end, lubricant must be removed from the lubrication system and then tested outside the lubrication system. However, this is very time-consuming. Furthermore, more precise information relating to the state of a lubricant could hitherto not be gathered without complex testing.

The object of the present disclosure is to provide a sensor device, a lubrication system and a method with which rapid, continuous and comprehensive monitoring of a lubricant is possible.

### SUMMARY

This object is achieved by a sensor device for detecting a state of a lubricant according to claim 1 and a lubrication system according to claim 11 and a method for detecting a state of a lubricant according to claim 12.

The sensor device may be used, for example, in a lubrication system, such as a central lubrication system. Such a lubrication system comprises a lubricant tank, a pump and at least one lubrication point, wherein the pump is designed to deliver lubricant from the lubricant tank to the at least one lubrication point. The lubrication system furthermore has one or more lubricant-conducting lines, also called lubrication lines, which connect the lubricant tank, the pump and the lubrication point. The pump may also be connected to multiple lubrication points via multiple lubrication lines. Furthermore, a lubrication line may lead from one or more lubrication points into a collection tank for used lubricant.

The lubrication points may be any parts of a mechanical system which need to be lubricated, e.g. bearings etc. Lubricant is delivered from the lubricant tank to the lubrication points as required. The pump is therefore provided in the lubrication line between the lubricant tank and lubrication point in order to bring about the transportation of the lubricant. The lubricant may be any lubricant in fluid or paste-like form, e.g. oil or grease.

To ensure that the lubricant used enables adequate lubrication of the lubrication point(s), the lubrication system may have a sensor device for detecting a state of the lubricant in the lubrication system, as described below.

Alternatively, the sensor device described below may be used in a stand-alone manner, independently of a lubrication system, for example in a laboratory environment. In this case, the lubricant to be tested may be introduced into the sensor device, e.g. in the form of a probe.

In both cases, a sensor device may be used, which comprises a sensor unit designed to measure an impedance of the lubricant according to the frequency of an applied alternating current. Furthermore, the sensor device has an evaluation unit, which is designed to determine a state of the lubricant based on the measured impedance.

When used in a lubrication system, the sensor unit may be arranged downstream of the lubricant tank and/or downstream of a pump in the lubricant-conducting line and/or it may be arranged upstream or downstream of a consumer, i.e. a corresponding lubrication point, and/or at the at least one lubrication point. Alternatively, the sensor unit may also be arranged in the pump and/or in the lubricant collection tank if the lubricant from the lubrication points is collected in such a lubricant collection tank. For the sake of simplicity, the sensor device is described in connection with a lubrication system below. However, it should be noted that the features described here also apply independently of a lubrication system, i.e. when used in a laboratory environment.

Through the above-described measurement of the impedance, it is possible to detect a change in the lubricant, in particular a deterioration of the lubricant. The impedance, also known as alternating current resistance, depends on the ratio of the applied electrical voltage to the flow of current through the lubricant. This is influenced by the state of the lubricant, e.g. the degree of contamination, water content, etc. This means that an increase or reduction in the impedance in turn allows it to be concluded that the state of the lubricant has changed. The measurement and monitoring of the impedance may therefore enable the state of the lubricant to be monitored in a simple manner.

The impedance may be determined, in particular, via so-called impedance spectroscopy. The impedance here is determined according to the frequency of an applied alternating current, wherein the impedance in the case of multiple frequencies is determined over a defined frequency range (spectrum). The impedance spectroscopy may be carried out in a known manner. For the sake of simplicity, impedance measurement is referred to below, wherein this is understood to be the measurement of the impedance via impedance spectroscopy according to the frequency of the alternating current.

In particular, the frequency range of 10 Hz to at least 5 MHz may be used for the impedance measurement. The measurement provides the resistive and capacitive components and the phase angle. Within this measurement range, specific characteristic values, such as minima/maxima, mean value, median, gradient or area, which may be used for analysis, may be ascertained from the respective curves. Different measuring voltages (e.g. 1, 2 or 5 volts) may provide further information.

The sensor device enables simple monitoring of the state of the lubricant through determination and evaluation of the impedance. The sensor device may, in particular, also be integrated in existing lubrication systems. Furthermore, in contrast with previous systems, real-time monitoring is possible, since lubricant does not need to be removed from the lubrication system and tested externally. Furthermore, the monitoring may take place continuously and is not dependent on probes which have to be removed from the lubrication system. Alternatively, it is possible to perform checks on the lubricant at certain intervals and not carry out continuous monitoring and measurement. As already mentioned above, the sensor device may also be used separately from a lubrication system, e.g. in a laboratory environment, and offers the option of obtaining more comprehensive information about the state of the lubricant through measurement of the impedance.

The sensor unit may be connected to the evaluation unit via cables in order to share the measured impedance with the evaluation unit. Alternatively, the sensor unit may communicate with the evaluation unit wirelessly, e.g. via radio. In the latter case, the sensor unit preferably comprises communication means for communicating with the evaluation unit and/or further electronics for relaying the measured impedance to the evaluation unit. The evaluation unit may thus be arranged at any point, even separately from the lubrication system or the sensor unit. The integration of the evaluation unit in a control unit of the lubrication system or other control or processing units is likewise possible.

According to one embodiment, the sensor unit has a housing having a supply opening and a discharge opening for supplying and discharging lubricant to the housing and from the housing. The sensor unit here is arranged in the lubricant-conducting line, for example. In order to achieve contact between the sensor unit and the lubricant, which is necessary for measuring the impedance, lubricant is introduced into the housing via the supply opening and the discharge opening of the housing, i.e. the lubricant is conveyed through the sensor unit. The housing here is preferably arranged in such a way that the lubricant is introduced into the housing, and discharged from the housing again, during its transportation through the lubrication circuit of the lubrication system. Alternatively, the sensor unit may also be arranged directly in lubricant, e.g. in the lubrication system, without a housing. Alternatively, part of the sensor device, e.g. the sensor unit, may also move and thereby shear the lubricant. The movement may be rotational or linear. A continuous movement or an oscillating movement is possible. As a result of the movement, an alignment of the molecules of the lubricant takes place, giving additional value to a static measurement and enabling new characteristic values.

Alternatively to the above-described dynamic measurement through relative movement of the lubricant, the state of the lubricant may also be detected statically, i.e. without the movement of the lubricant relative to the sensor unit.

According to a preferred embodiment, the sensor unit has at least two electrodes, which are arranged in contact with the lubricant. The electrodes may be arranged in various ways, e.g. as plate electrodes, tube electrodes or interdigital electrodes. Other arrangements of electrodes are likewise possible. The electrodes may be designed either with a conductive surface, e.g. in order to enable resistive and capacitive measurement, or they may be insulated with respect to the lubricant in order to enable capacitive measurement. In addition, the sensor unit may have a current source, which is connected to the electrodes in order to apply alternating current to the electrodes. By applying the alternating current, it is possible to measure the impedance spectroscopy for measuring the impedance of the lubricant according to different frequencies.

According to a further embodiment, the at least two electrodes are arranged at a predefined spacing from one another. The spacing between the two electrodes may depend on the quantity and/or type of lubricant. If the lubricant is more liquid, the electrodes may be arranged closer to one another; if it is more viscous, the electrodes need to be at a greater spacing from one another to allow the lubricant to pass through. It is also possible to set the spacing between the electrodes mechanically. This may be realized via corresponding elements, e.g. levers or piezo elements, which are excited accordingly in order to alter the spacing between the electrodes. In each case, to enable reliable and accurate measurement, the spacing during the measurement of the impedance should not change.

According to a further embodiment, the evaluation unit is designed to compare the measured impedance with a reference value. If the measured impedance deviates from the reference value, this may indicate a change in the properties of the lubricant. Such a change in the lubricant may influence the lubrication properties of the lubricant. This may be, for example, water or moisture finding its way into the lubricant, contaminants in the lubricant, e.g. abrasion debris from mechanical parts, a change in pressure or the like. If, for example, iron is contained in the lubricant due to abrasion or the lubricant contains more water than previously, this leads to a change in the value of the impedance and therefore to a deviation from the reference value.

If such a deviation from the reference value is established, the evaluation unit may output a corresponding warning signal, e.g. to a control unit or display unit, in order to notify the user of a deterioration of the lubricant. This notification may indicate that a maintenance measure is required or that that a maintenance measure might be required in the (near) future. Such a maintenance measure may be a lubricant change, for example.

It is also possible to not only establish a deviation from the reference value but, at the same time, to also draw conclusions about the nature of the change in the state of the lubricant through the nature of the deviation. Furthermore, the evaluation unit may not only establish a deviation from the reference value in general, but it may also output more comprehensive measurement value information via which further conclusions are possible. For example, the quantity of abrasion debris or the quantity of water in the lubricant may be determined from the extent of the deviation. Furthermore, it is possible that, in the event of a deviation of the measured impedance from a predefined reference value, the evaluation unit establishes whether the correct lubricant is present in the lubrication system or in the lubricant tank.

According to a further embodiment, the sensor unit is designed to measure the impedance of the lubricant in an initial state of the lubricant, i.e. of new lubricant, and to store this as a reference value. In this way, a reference value may be ascertained at the start of the operation of the lubrication system with the new lubricant and may furthermore be used to monitor the state. It is assumed here that the lubricant in its new state is in a virtually ideal state, i.e. correct moisture content, no abrasion debris, no other contaminants, etc. During further operation of the lubrication system, it is then possible to monitor whether the state of the lubricant changes or deteriorates in comparison with this initial ideal state. A comparison with other reference values from alternative systems or other databases is unnecessary in this case. Since the determination of the reference value may be carried out in a new state of the lubricant, this is also possible in lubrication systems which are retrofitted with such a sensor device.

Alternatively, it is possible for measurements from other lubrication systems containing similar or comparable lubricants to be used as the reference value. These measurements may be stored in a database and retrieved from this for comparison by the evaluation unit. This is particularly advantageous if an existing lubrication system is retrofitted with such a sensor device, since it is possible to refer back to existing information here and it is not necessary to determine a reference value in the said existing lubrication system.

According to a further embodiment, the sensor device has a temperature-control unit, which is designed to regulate the temperature of the lubricant—at least in the sensor unit—to a predetermined temperature. Since the impedance of the lubricant depends on the current temperature of the lubricant, it is possible, according to this embodiment, to regulate the temperature of the lubricant to a predetermined temperature in order to enable the conductivity measurement to be carried out at this predetermined temperature. The evaluation unit may then compare the measured impedance with a reference value, wherein the reference value corresponds to the impedance of a reference lubricant at the predetermined temperature. For example, a reference value with an associated temperature may firstly be retrieved from a database and the temperature-control unit may then regulate the temperature of the lubricant to the corresponding associated temperature before the impedance measurement then takes place.

The temperature-control unit may be arranged in the sensor unit, e.g. in a housing of the sensor unit, and may regulate the temperature of the lubricant introduced into the sensor unit for measurement purposes accordingly before the impedance is measured. Alternatively, the temperature-control unit may also be arranged such that it may regulate the temperature of the lubricant, for example in the lubricant-conducting line or in the pump, accordingly. The temperature-control unit may comprise a heating and/or cooling element, for example.

According to a further embodiment, the sensor device has a temperature sensor, which is designed to measure a temperature of the lubricant. The evaluation unit in this case is designed to determine the state of the lubricant based on the measured impedance and the measured temperature. In particular, the evaluation unit may compare the measured impedance with a stored reference value which is associated with the same temperature. For example, various impedance values with associated temperatures may be stored in a database and the evaluation unit may retrieve the corresponding reference value with the associated temperature from the database. The temperature sensor may be arranged in the sensor unit itself or it may be arranged in the vicinity of the sensor unit, for example in the lubricant-conducting line. The temperature sensor may be any temperature sensor which is capable of determining the temperature of the lubricant.

Via the sensor device proposed here, simple monitoring of the lubricant in a lubrication system is possible, even for inexperienced users. The sensor unit may be easily integrated in existing lubrication systems and the evaluation unit may be arranged outside the lubrication system, but in communication with the sensor unit. The reference values may come from other sources, e.g. similar lubrication systems, historical data, comparator systems, etc. Alternatively, a reference measurement for the specific application system is possible, as has already been described.

According to a further aspect, a lubrication system having a pump and at least one lubrication point is proposed, wherein the pump is designed to deliver lubricant from the lubricant tank to the at least one lubrication point. The lubrication system furthermore comprises one or more lubricant-conducting lines, which connect the lubricant tank, the pump and the lubrication point, and a sensor device, as described above, which may be arranged in one or more of the lubricant-conducting lines, in the pump and/or in a lubricant collection tank and/or at the at least one lubrication point.

Furthermore, a method for detecting a state of a lubricant is proposed. The method comprises the following steps: measuring the impedance of the lubricant according to the frequency of an applied alternating current and determining a state of the lubricant based on the measured impedance.

Further advantages and advantageous embodiments are indicated in the description, the drawings and the claims. In particular, the combinations of features indicated in the description and in the drawings are purely exemplary, which means that the features may also be present individually or combined in another way.

### BRIEF DESCRIPTION OF THE DRAWINGS

The present disclosure shall be explained in more detail below with reference to exemplary embodiments illustrated in the drawings. The exemplary embodiments and the combinations shown in the exemplary embodiments are purely exemplary and are not intended to stipulate the scope of protection of the present disclosure. This is defined by the appended claims.

In the drawings:

FIG. 1 shows a schematic partial view of a lubrication system with a sensor device;

FIG. 2 shows a schematic partial view of the lubrication system of FIG. 1 with a variant of the sensor device; and

FIGS. 3a-3c show various embodiments of a sensor unit of the sensor device of FIGS. 1 and 2.

### DETAILED DESCRIPTION

Equivalent or functionally equivalent elements are denoted by the same reference signs below.

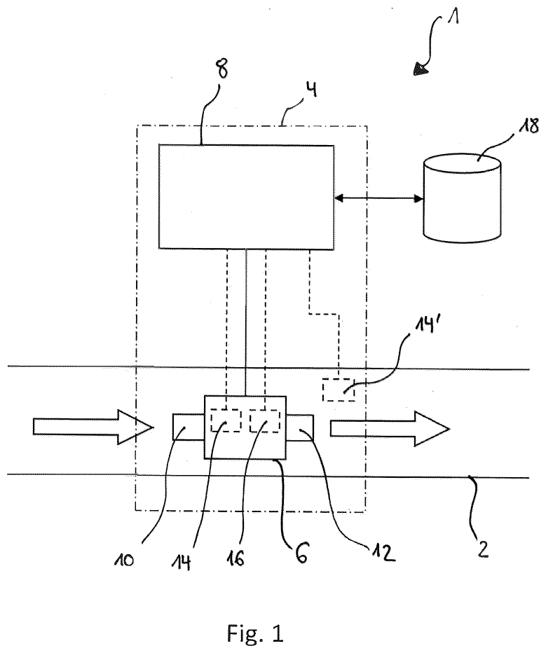

FIG. 1 shows part of a lubrication system 1. The lubrication system 1 has a lubricant tank (not shown), a pump, at least one lubrication point or consumer (not shown) and one or more lubricant-conducting lines 2, which connect the lubricant tank, the pump and/or the lubrication points. The lubricant-conducting line 2 may also lead from the lubrication point to a lubricant collection tank. Since the lubricant in such a system 1 may change during operation due to diverse external influences, e.g. abrasion at the lubrication point or due to moisture or water making its way into the lubricant, it is necessary to monitor the state of the lubricant in such lubrication systems 1. A sensor device 4 is provided for this purpose. It should be noted that, although the sensor device 4 is described in combination with a lubrication system 1 below, the sensor device 4 may also be used separately from the lubrication system 1, for example in a laboratory environment, in an analogous manner.

The sensor device 4 has a sensor unit 6 and an evaluation unit 8. The sensor unit 6 is arranged in the lubricant-conducting line 2 in the embodiment illustrated in FIG. 1. Alternatively, the sensor unit 6 may also be arranged in the pump, at the lubrication point and/or in the lubricant collection tank.

The sensor unit 6 may be connected to the evaluation unit 8 via a cable. Alternatively, the sensor unit 6 and the evaluation unit 8 may also communicate wirelessly, e.g. via radio communication. In this case, the sensor unit 6 preferably has further electronics, e.g. communication means 6, in order to communicate with the evaluation unit 8. This is advantageous in that the evaluation unit 8 may be arranged separately from the sensor unit 6 and, for example, also combined with a control unit of the lubrication system 1 or other processing units 1.

The sensor unit 6 may have a housing, which has a supply opening 10 and a discharge opening 12. Lubricant may be transported into the sensor unit 6 via the supply opening 10 and the lubricant may be conducted out of the sensor unit 6 again via the discharge opening 12. The sensor unit 6 is preferably arranged such that the supply opening 10 and the discharge opening 12 correspond to the flow direction of the lubricant, as indicated by the arrows.

The sensor unit 6 measures an impedance of the lubricant. To this end, in particular, impedance spectroscopy is used. The measured impedance is then transmitted to the evaluation unit 8 by the sensor unit 6. The evaluation unit 8 may then compare the measured impedance with a reference value, which is stored, for example, in a database 18. The reference value may be a reference value of a comparable lubrication system, wherein a plurality of reference values for different lubrication systems may be stored in the database. Alternatively, the sensor unit 6 may also perform a reference measurement at the start of the operation of the lubrication system 1 and store the result of this reference measurement in the database 18 as a reference value.

Since the impedance of the lubricant also depends on the temperature, the sensor device 4 may have a temperature sensor 14, 14′. This temperature sensor 14, 14′ may either be arranged directly in the sensor unit 6 or it may be arranged in the lubricant-conducting line 2 or in the lubricant tank. The temperature sensor 14, 14′ may also be arranged in multiple positions. It is preferably arranged at a point at which the impedance of the lubricant is measured. If a temperature of the lubricant is measured, the evaluation unit 8 may also take into account the temperature for comparing the measured impedance with a reference value. This means that the evaluation unit 8 uses a reference value which corresponds to a reference lubricant at the same temperature.

Instead of, or in addition to, such a temperature sensor 14, 14′, the sensor device 4 may also have a temperature-control unit 16. In this case, the temperature-control unit 16 may not only measure the temperature of the lubricant, but also regulate the temperature of the lubricant to a certain temperature. If the reference values are for a lubricant at a predetermined temperature, the temperature-control unit 16 may bring the lubricant in the lubricant-conducting line 2 up or down to this temperature, for example, and the evaluation unit 8 may then compare the impedance measured at the correct temperature with the corresponding reference value.

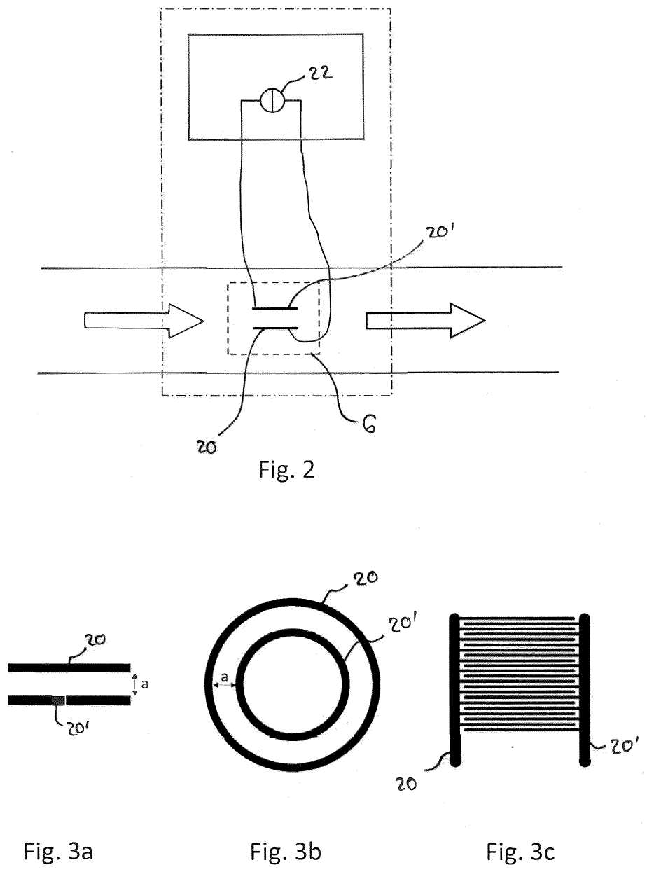

The sensor unit 6 preferably has electrodes 20, 20′ for measuring the impedance, as shown in FIG. 2. The spacing a between the two electrodes 20, 20′ may be adapted to support different types of lubricant, for example.

To measure the impedance, the electrodes 20, 20′ are connected to an alternating current source 22. In the embodiment shown in FIG. 2, the current source 22 is arranged in the evaluation unit 8. However, the current source 22 may also be arranged separately from this, for example in the sensor unit 6.

Via the current source 22, an alternating current is applied to the electrodes 20, 20′ and, on the basis of this, the impedance spectroscopy is then carried out. The electrodes 20, 20′ may be used in various configurations. As shown in FIG. 2 and FIG. 3a, the two electrodes 20, 20′ may be present as plate electrodes. Alternatively, the two electrodes 20, 20′ may also be present as tube electrodes (see FIG. 3b). Another variant, as shown in FIG. 3c, consists of an interdigital electrode, wherein the two electrodes 20, 20′ are designed in the shape of a comb and mesh with one another.

The mutual spacing a of the electrodes 20, 20′ may be selected as desired and, in some embodiments, may also be adapted according to the lubrication system 1 used. For example, the spacing a may be increased if the lubricant is less liquid, as is the case with grease, for example, and may be decreased if the lubricant is more liquid, e.g. in the case of oil.

Via the sensor device 4, it is possible to make various statements about the lubricant based on the measured impedance. In particular, when compared with stored reference values, it is possible to determine the lubricant type, the quality of the lubricant, the presence of contaminants (e.g. abrasion debris) therein, the presence of water therein, the ageing state, the degeneration of the lubricant, the additive content and/or further features.

Via the sensor device described here, a simple option is therefore provided for determining and monitoring the state of a lubricant. The sensor device here may be integrated, in particular, in already existing lubrication systems and provides a user with comprehensive information about the state of the lubricant in a simple manner, without further prior knowledge.

### LIST OF REFERENCE SIGNS

1 Lubrication system
2 Lubricant-conducting line
4 Sensor device
6 Sensor unit
8 Evaluation unit
10 Supply opening
12 Discharge opening
14, 14 Temperature sensor unit
16 Temperature-control unit
18 Database
20, 20 Electrodes
22 Current source
a Spacing

What is claimed is:

1. A sensor device for detecting a state of a lubricant, the sensor device comprising:
a sensor unit configured to measure the impedance of the lubricant according to the frequency of an applied alternating current;
an evaluation unit configured to determine a state of the lubricant based on the measured impedance.

2. The sensor device according to claim 1, wherein the sensor unit has a housing having a supply opening and a discharge opening for supplying and discharging lubricant to the housing and from the housing.

3. The sensor device according to claim 1, wherein the sensor unit has at least two electrodes arranged to contact with the lubricant.

4. The sensor device according to claim 3, wherein the sensor unit has an alternating current source, which is connected to the electrodes in order to apply alternating current to the electrodes.

5. The sensor device according to claim 1, wherein the evaluation unit is configured to compare the measured impedance with a reference value.

6. The sensor device according to claim 5, wherein the sensor unit is configured to measure the impedance of the lubricant in an initial state of the lubricant, and to store this as a reference value.

7. The sensor device according to claim 1, wherein the sensor device has a temperature-control unit, and wherein the temperature-control unit is designed to regulate the temperature of the lubricant, at least in the sensor unit, to a predetermined temperature.

8. The sensor device according to claim 7, wherein the evaluation unit is designed to compare the measured impedance with a reference value, wherein the reference value corresponds to the impedance of a reference lubricant at the predetermined temperature.

9. The sensor device according to claim 1, wherein the sensor device has a temperature sensor, which is designed to measure a temperature of the lubricant, and wherein the evaluation unit is designed to determine the state of the lubricant based on the measured impedance and the measured temperature.

10. The sensor device according to claim 1, wherein the sensor device is part of a lubrication system, in particular a central lubrication system, or wherein the sensor device is arranged separately from a lubrication system in a laboratory environment.

11. The sensor device according to claim 2, wherein the sensor unit has at least two electrodes arranged to contact with the lubricant.

12. The sensor device according to claim 11, wherein the sensor unit has an alternating current source, which is connected to the electrodes in order to apply alternating current to the electrodes.

13. The sensor device according to claim 12, wherein the evaluation unit is configured to compare the measured impedance with a reference value.

14. The sensor device according to claim 13, wherein the sensor unit is configured to measure the impedance of the lubricant in an initial state of the lubricant, and to store this as a reference value.

15. The sensor device according to claim 14, wherein the sensor device has a temperature-control unit, and wherein the temperature-control unit is designed to regulate the temperature of the lubricant, at least in the sensor unit, to a predetermined temperature.

16. The sensor device according to claim 15, wherein the evaluation unit is designed to compare the measured impedance with a reference value, wherein the reference value corresponds to the impedance of a reference lubricant at the predetermined temperature.

17. The sensor device according to claim 16, wherein the sensor device has a temperature sensor, which is designed to measure a temperature of the lubricant, and wherein the evaluation unit is designed to determine the state of the lubricant based on the measured impedance and the measured temperature.

18. The sensor device according to claim 17, wherein the sensor device is part of a lubrication system, in particular a central lubrication system, or wherein the sensor device is arranged separately from a lubrication system in a laboratory environment.

19. A lubrication system comprising:
at least one lubrication point;
a lubricant tank;
a pump configured to deliver lubricant from the lubricant tank to the at least one lubrication point;
one or more lubricant-conducting lines connecting the lubricant tank, the pump and the at least one lubrication point;
a sensor device according to claim 1, the sensor device being arranged in one or more of the lubricant-conducting lines, in the pump, in a lubricant collection tank, and/or at the at least one lubrication point.

20. A method for detecting a state of a lubricant comprising:
measuring the impedance of the lubricant according to the frequency of an applied alternating current; and
determining a state of the lubricant based on the measured impedance.
